(19) United States
(12) Patent Application Publication
(10) Pub. No.: US 2026/0263433 A1
(43) Pub. Date: Sep. 10, 2026

(54) TETRAZOLIUM AND FORMAZAN COMPOUNDS AND METHODS OF MAKING AND USING THE SAME

(71) Applicant: ARIZONA BOARD OF REGENTS ON BEHALF OF THE UNIVERSITY OF ARIZONA, Tucson, AZ (US)

(72) Inventors: Elisa TOMAT, Tucson, AZ (US); Zoufeng XU, Tucson, AZ (US); Yu-Shien SUNG, Tucson, AZ (US)

(21) Appl. No.: 19/099,731

(22) PCT Filed: Aug. 10, 2023
(86) PCT No.: PCT/US2023/029921

§ 371 Date: Jan. 29, 2025

Related U.S. Application Data
(60) Provisional application No. 63/370,998, filed on Aug. 10, 2022.

(51) Int. Cl. A61K 31/4439; A61K 31/427; A61K 31/428; A61K 31/4709; A61K 31/4725; A61K 31/498; A61K 31/517; A61P 35/00

(52) U.S. Cl. CPC A61K 31/4439; A61K 31/427; A61K 31/428; A61K 31/4709; A61K 31/4725; A61K 31/498; A61K 31/517; A61P 35/00

(57) ABSTRACT

Tetrazolium-based compounds are described herein that are capable of undergoing reduction reactions under physiological conditions after crossing over the cell membrane. Reduction of the tetrazolium-based compounds results in ring-opening to form formazan chelators that bind to iron, copper, and other metals present in the body. Iron chelation in particular has beneficial antiproliferative effects, and the compounds of the present invention are therefore useful in treating proliferative conditions such as cancer as well as iron overload conditions.

### CROSS-REFERENCE TO RELATED APPLICATIONS

This application claims priority to U.S. Provisional Application No. 63/370,998, filed Aug. 10, 2022, the entire disclosure of which is hereby incorporated by reference.

This invention was made with government support under Grant No. GM127646 from the National Institutes of Health. The U.S. government has certain rights in the invention.

### FIELD OF THE INVENTION

The inventions described herein relate to tetrazolium compounds that are capable of forming formazan compounds under in vivo conditions, formazan compounds that are capable of binding to metal ions in vivo, and methods of making and using the same to treat various disorders and diseases.

### BACKGROUND OF THE INVENTION

Altered metabolism and homeostasis of transition metal ions (such as iron, copper, and zinc) are associated with several pathological conditions including cancer^{1,2} and neurodegeneration.³ Thus, metal-binding compounds (chelators) may play important roles in these pathways and may be potential drug candidates for these pathological conditions.⁴⁻⁷ One such example is the use of disulfide switches to mask the tridentate binding unit of thiosemicarbazone and aroylhydrazone chelators, which, upon cellular uptake, in vivo reduction of the disulfide bond releases a thiolate chelator that coordinates iron with high affinity in mammalian cells.⁹⁻¹⁴

### BRIEF SUMMARY OF THE INVENTION

The present invention is directed to tetrazolium compounds and formazan compounds disclosed herein having utility in the chelation, sequestration, and/or removal of excess iron from the body, as well as in the treatment of disorders having an etiology associated with excess metal(s) in the body and/or cells, such as various disorders associated with excess iron or iron overload, as well as hyperproliferative disorders such as various cancers.

The present invention is directed to a new class of prochelator compounds based on the reduction of tetrazolium ions in mammalian cells to form a formazan compound under in vivo conditions. Following cellular uptake, the tetrazolium compounds of the present invention undergo reduction to produce metal-binding formazan species that are capable of in vivo binding of various metals. In this approach, the prochelator compound forms an active metal-binding chelator under specific conditions in order to minimize indiscriminate depletion of essential metal ions and potential side effects.⁸ The resulting formazan compounds are stable in blood serum and present antiproliferative activities in the 10-30 μM range, as confirmed against a panel of various cancer cell lines. Multiple cell-based assays confirmed the intracellular activation of these prochelators and their ability to interfere with iron availability. As such, the present invention is directed to the tetrazolium compounds as well as the formazan compounds disclosed herein and/or a formazan-metal complex. The present invention is also directed to methods of treating a disorder associated with an excess of metal in a subject in need thereof comprising administering a compound of Formulas I-IV (as described herein) to the subject. The present invention is directed to methods of treating iron overload disorder, iron dysregulation, thalassemia, hemochromatosis, myelodysplasia, Alzheimer's disease, and/or Parkinson's disease. The present invention is also directed to methods of inhibiting the proliferation of cancer cells. The present invention is also directed to methods of treating hyperproliferative disorders such as various cancers. The present invention is also directed to pharmaceutical compositions comprising the tetrazolium compounds disclosed herein.

### BRIEF DESCRIPTION OF THE FIGURES AND DRAWING

FIG. 1 depicts the reduction of the tetrazolium 5-(4,5-dimethylthiazol-2-yl)-1,3-diphenyltetrazolium bromide (“MTT”) ion to formazan MTF, which is accompanied by a color change from yellow to purple and is the basis for the widely employed MTT assay of cell proliferation and viability.

FIG. 2 depicts the synthesis and crystal structure of Fe(MTF-H)2.

FIGS. 3A and 3B depict exemplary formazan compounds of the present disclosure.

FIG. 3C depicts the synthesis of the formazan scaffolds of the present disclosure.

FIGS. 4A and 4B depict the structures of the tetrazolium scaffolds of the present disclosure.

FIG. 4C depicts the synthesis of the tetrazolium scaffolds of the present disclosure.

FIGS. 5A and 5B depict the assessment of stability of the tetrazolium-based prochelators in fetal bovine serum (FBS) for a period of 24 h.

FIGS. 6A-6B depict the effects of iron supplementation on toxicity in A2780 ovarian cancer cells. The percentage of dead cells was determined using the LIVE/DEAD fixable stain as measured by flow cytometry. FIG. 6A depicts the quantification of dead cells after treating in the presence of test compounds (40 μM) with or without ferric ammonium citrate (FAC, 50 μM) for 48 h. FIG. 6B depicts the quantification of dead cells after treating in the presence of test compounds (40 μM) with or without holo-transferrin (holo-TF, 50 μM) for 48 h. Experiments were conducted in triplicate and the values shown are averages ±standard deviation. **p<0.01, ***p<0.001, ****p<0.0001.

### DETAILED DESCRIPTION

Throughout this disclosure, the description refers to certain compounds, compositions and methods of using the compounds and/or compositions. Where the disclosure describes or claims a feature or embodiment associate with a compound and/or composition, such a feature or embodiment is equally applicable to the methods of using the compounds and/or compositions. Likewise, where the disclosure describes or claims a feature or embodiment associated with a method of using a compound and/or composition (e.g., a method of treatment, method of administration, and/or other method of use), such a feature or embodiment is equally applicable to the compounds and compositions disclosed herein.

Reference to values stated in ranges include each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “composition” can refer to either a tetrazolium and/or formazan compound as described herein alone or in combination with other ingredients. In some embodiments, the compositions comprise at least a portion of the compounds disclosed herein in neutral or cationic form, or as a salt, hydrate, solvate, or complex.

As used herein, “pharmaceutically acceptable” refers to a carrier, diluent, adjuvant, or excipient that is compatible with the compounds of the present invention and the other ingredients of a pharmaceutical formulation such that the composition is suitable for pharmaceutical use and not deleterious to the recipient thereof.

As used herein, “administering” refers to providing a compound described herein to a subject in need thereof by oral, intravenous, transdermal, transmucosal, or other means that enables the compounds of the present invention to have systemic effect or to have a targeted effect on a specific location or group of cells in the body. “Administering” a composition may be accomplished by, for example, injection, oral administration, topical administration, or by these methods in combination with other known techniques. Such combination techniques include heating, radiation, ultrasound and the use of delivery agents.

A “therapeutically effective amount” or “effective amount” of a compound or composition of the present invention is a predetermined amount calculated to achieve a desired effect. For example, a desired effect can be to inhibit, block, or reverse the activation, migration, proliferation, or alteration of cellular function, while preserving the normal function of normal (e.g., non-cancerous) cells. A “therapeutically effective amount” or “effective amount” of a compound or composition of the present invention can also refer to a predetermined amount of a compound or composition of the present invention required to chelate a specific amount of a metal under in vivo or physiologic conditions. The activity contemplated by the compounds, compositions, and methods described herein includes both medical therapeutic and/or prophylactic treatment, as appropriate, and the compounds, compositions, and methods of the invention can be used to provide improvement in any of the conditions described. It is also contemplated that the compounds and compositions described herein may be administered to healthy subjects or individuals not exhibiting symptoms but who may be at risk of developing a particular disorder. The specific dose of a compound administered according to this invention to obtain therapeutic and/or prophylactic effects will, of course, be determined by the particular circumstances surrounding the case, including, for example, the compound administered, the route of administration, and the condition being treated. However, it will be understood that the chosen dosage ranges are not intended to limit the scope of the invention in any way. A therapeutically effective amount of compound of this invention is typically an amount such that when it is administered a pharmaceutically acceptable excipient and/or composition, it is sufficient to achieve an effective systemic concentration or local concentration in the tissue.

The terms “treat,” “treated,” and/or “treating” as used herein refer to both therapeutic treatment and prophylactic or preventative measures, wherein the object is to prevent or slow down (lessen) an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease.

The term “subject,” as used herein, describes an organism, including mammals, to which treatment with the compositions and compounds according to the subject disclosure can be administered. Mammanlian species that can benefit from administration of the compounds and compositions of the present invention by the disclosed methods include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

In some embodiments, the present invention is directed to a tetrazolium compound of Formula I:

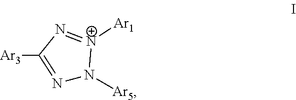

wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

In some embodiments, Ar₁, Ar₃, and/or Ar₅ in the compounds of Formulas I-IV are metal-binding donors. As such, in some embodiments at least of Ar₁, Ar₃, or Ar₅ is optionally substituted 2-pyridiyl, 3-pyridyl, or 4-pyridyl, optionally substituted 2-hydroxyphenyl, 3-hydroxyphenyl, or 4-hydroxyphenyl, optionally substituted 2-carboxyphenyl, 3-carboxyphenyl, or 4-carboxyphenyl, optionally substituted 2-quinolinyl, 3-quinolinyl, or 4-quinolinyl, optionally substituted imidazolyl., or a combination thereof.

In some embodiments, the present invention is directed to compounds of Formula I, wherein Ar₁ is optionally substituted phenyl, and Ar₃ and Ar₅ are selected from the list of optionally substituted functional groups listed above.

In some embodiments, the present invention is directed to compounds of Formula I, wherein Ar₁ is optionally substituted phenyl; Ar₃ is selected from: optionally substituted phenyl, optionally substituted 2-pyridyl, optionally substituted 2-thiazolyl, and optionally substituted 2-benzothiazolyl; and Ar₅ is selected from the list of optionally substituted functional groups listed above.

In some embodiments, the present invention is directed to compounds of Formula I wherein Ar₁ is optionally substituted phenyl; Ar₃ is selected from: optionally substituted phenyl, optionally substituted 2-pyridyl, optionally substituted 2-thiazolyl, and optionally substituted 2-benzothiazolyl; and Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

In some embodiments, the present invention is directed to compounds of Formula I wherein Ar₁ is unsubstituted phenyl; Ar₃ is selected from unsubstituted phenyl, 2-hydroxyphenyl, and 2-pyridyl; and Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

In some embodiments, the present invention is directed to a tetrazolium compound of Formula II:

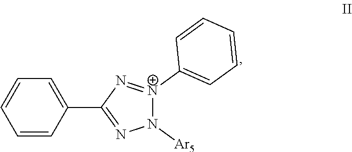

wherein Ar₅ is selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

In some embodiments, the present invention is directed to compounds of Formulas II wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

In some embodiments, the present invention is directed to compounds of Formula II wherein Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

In some embodiments, the present invention is directed to compounds of Formula I or II selected from:

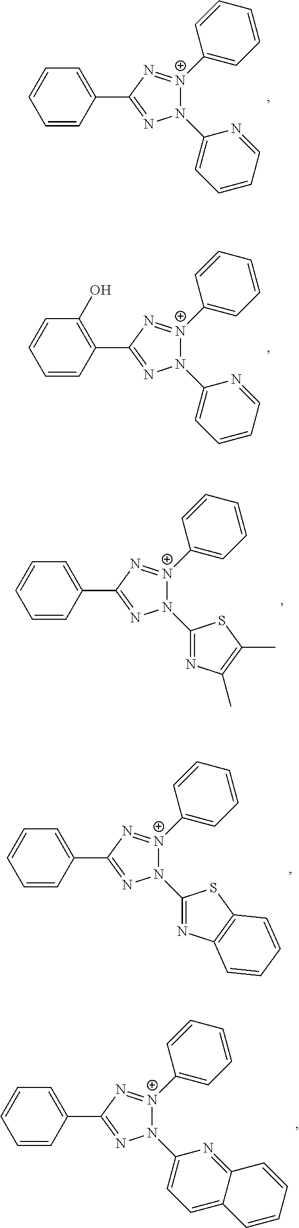 [SMILES: c1ccc(-c2nn(-c3ccccn3)[n+](-c3ccccc3)n2)cc1 ; Oc1ccccc1-c1nn(-c2ccccn2)[n+](-c2ccccc2)n1 ; Cc1nc(-n2nc(-c3ccccc3)n[n+]2-c2ccccc2)sc1C ; c1ccc(-c2nn(-c3nc4ccccc4s3)[n+](-c3ccccc3)n2)cc1 ; c1ccc(-c2nn(-c3ccc4ccccc4n3)[n+](-c3ccccc3)n2)cc1]

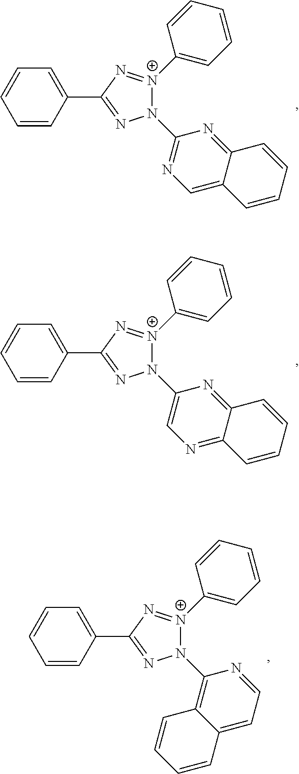 [SMILES: c1ccc(-c2nn(-c3ncc4ccccc4n3)[n+](-c3ccccc3)n2)cc1 ; c1ccc(-c2nn(-c3cnc4ccccc4n3)[n+](-c3ccccc3)n2)cc1 ; c1ccc(-c2nn(-c3nccc4ccccc34)[n+](-c3ccccc3)n2)cc1]

and combinations thereof.

The preferred compounds of Formulas I and II undergo reduction under physiologic conditions. In some embodiments, the compounds of Formulas I and II undergo reduction at about 0.1 V to about −0.5 V (relative to the Normal Hydrogen Electrode, “NHE”). Upon reduction under physiologic conditions, the preferred compounds of Formulas I or II undergo a ring-opening reaction to form a corresponding formazan compound. In some embodiments, the ring-opening is reversible. A compound of Formulas I or II can also undergo an irreversible ring-opening reaction.

The present invention is also directed to a new family of formazan compounds described herein. In some embodiments, the formazan compounds of the present invention can be formed in vivo by the intracellular reduction of a tetrazolium compound as disclosed herein, for example, through the activity of a biological cofactor such as NADH or NADPH.

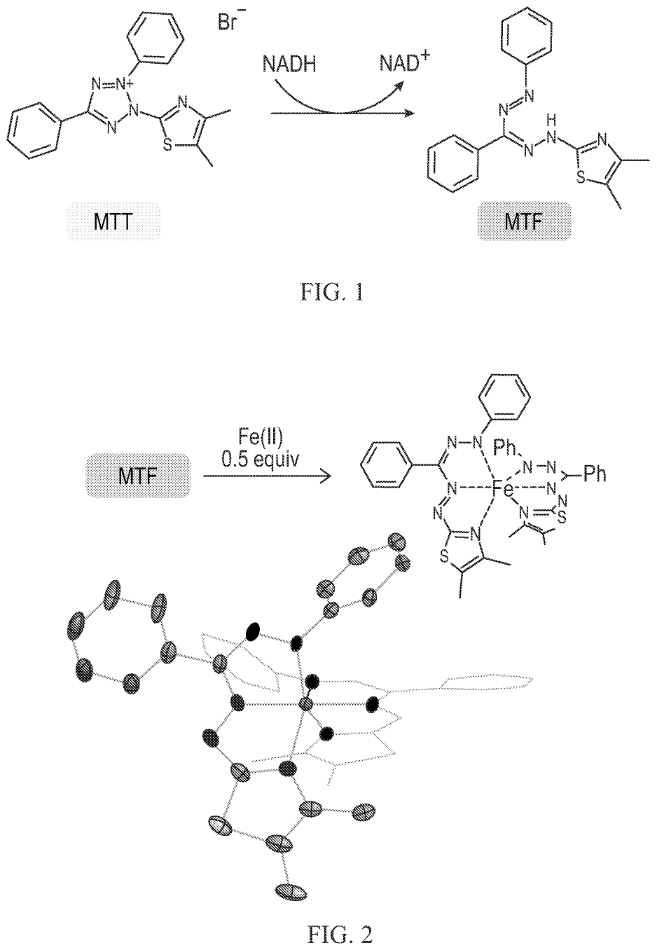

FIG. 1 provides a graphic representation of the reduction process for the formation of a formazan compound from MTT.

In some embodiments, the present invention is directed to a formazan compound of Formula III:

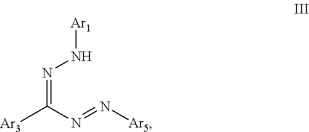

wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

In some embodiments, the present invention is directed to compounds of Formula III, wherein Ar₁ is optionally substituted phenyl, and Ar₃ and Ar₅ are selected from the list of optionally substituted functional groups listed above.

In some embodiments, the present invention is directed to compounds of Formula III, wherein Ar₁ is optionally substituted phenyl; Ar₃ is selected from: optionally substituted phenyl, optionally substituted 2-pyridyl, optionally substituted 2-thiazolyl, and optionally substituted 2-benzothiazolyl; and Ar₅ is selected from the list of optionally substituted functional groups listed above.

In some embodiments, the present invention is directed to compounds of Formula III, wherein Ar₁ is optionally substituted phenyl; Ar₃ is selected from: optionally substituted phenyl, optionally substituted 2-pyridyl, optionally substituted 2-thiazolyl, and optionally substituted 2-benzothiazolyl; and Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

In some embodiments, the present invention is directed to compounds of Formula III wherein Ar₁ is unsubstituted phenyl; Ar₃ is selected from unsubstituted phenyl, 2-hydroxyphenyl, and 2-pyridyl; and Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

In some embodiment, the present invention is directed to a formazan compound of Formula IV:

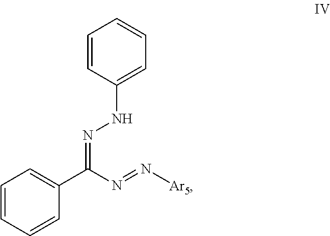

wherein Ar₅ is selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

In some embodiments, the present invention is directed to compounds of Formula IV, wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

In some embodiments, the present invention is directed to compounds of Formula IV, wherein Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

In some embodiments, the above compounds of Formulas I-IV are present as a salt, hydrate, solvate, and/or complex. When present as a salt, the salt forms of the compounds of the present invention can be prepared by any methods known to persons of ordinary skill in the art. Any pharmaceutically acceptable salts can be used. Any anion known to form a stable salt with a nitrogen-containing compound can be used.

In some embodiments, a compound of Formulas I-IV is present as an acid addition salt. Suitable pharmaceutically-acceptable acid addition salts of compounds within the scope of the present invention may be prepared from anions derived from inorganic or organic acids, or a combination thereof. Examples of salts derived from inorganic acids include but are not limited to chloride, bromide, iodide, nitrite, nitrate, carbonate, sulfate, phosphate, tetrafluoroborate, and combinations thereof. Suitable organic acids include aliphatic, cycloaliphatic, aromatic, araliphatic, heterocyclic, carboxylic, sulfonic, and combinations thereof. Representative organic acids include but are not limited to formate, acetate, propionate, succinate, glycolate, gluconate, lactate, malate, tartrate, citrate, ascorbate, glucuronate, maleate, fumarate, pyruvate, aspartate, glutamate, benzoate, anthranilate, mesylate, salicylate, 4-hydrobenzoate, phylacetate, mandelate, embonate, methanesulfonate, ethanesulfonate, benzenesulfonate, pantothenate, 2-hydroxyethanesulfonate, toluenesulfonate, sulfanilate, cyclohyexylaminosuflonate, stearate, algenate, hydrobutyrate, galactarate and galactumoate, and combinations thereof. In some preferred embodiments, a compound of Formulas I-IV as described herein are present as a bromide salt.

The present invention also encompasses optical isomers of the compounds of Formulas I-IV disclosed herein that can be obtained by resolution of a racemic mixture according to conventional processes, for example by formation of diastereoisomeric salts followed by separation of the mixture of diastereoisomers by crystallization. Other suitable methods for preparing isolated enantiomers of the disclosed compounds include distillation, crystallization, sublimation, and chromatographic separation using a chiral column.

Optically active compounds within the scope of the present invention can also be obtained by utilizing optically active starting materials. The isomers may be in the form of a free acid, a free base, an ester or a salt.

Hydrates and/or solvates of the compounds of the present invention may be stoichiometric as according to the mole ratio of the water or solvent molecule to the compound or salt thereof. Crystalline hydrates as disclosed herein may also be non-stoichiometric depending on the conditions of the unit cell which result in a thermodynamically or kinetically stable crystal. Crystalline salts and co-crystals may also be stoichiometric or non-stoichiometric for reasons stated above. One of ordinary skill in the art of crystallography understands that the components in the unit cell of a crystal may or may not be stoichiometric depending on the conditions that stabilize a crystal.

In some embodiments, the present invention is directed to a composition comprising a complex between a formazan compound of Formula III or IV as disclosed herein and a metal ion. Suitable metal ions present in complexes with a formazan compound of Formula III or IV include any metal found in the body whether under normal (i.e., healthy) condition or under a condition related to an excess of a metal in the body. In some embodiments, the metal ion present in a complex with a formazan compound of Formula III or IV is a transition metal, a rare earth metal, a main group metal, or a combination thereof. In some embodiments, the metal ion in a complex with a formazan compound of Formula III or IV is selected from: iron, copper, zinc, manganese, chromium, molybdenum, silver, cadmium, mercury, lead, arsenic, or a combination thereof. In preferred embodiments, the metal is iron.

The formazan-metal complexes of the present invention can be formed in vitro or in vivo. In preferred embodiments, the complex between a formazan compound of Formula III or IV as disclosed herein and a metal ion is formed in vivo.

FIG. 2 provides a graphic representation of a 2:1 ligand to metal complex formed between MTF and iron(II). Other stoichiometric ratios are contemplated within the scope of the present invention, as a person of ordinary skill in the art would understand. In some embodiments, the present invention is directed to a 2:1 ligand to metal complex formed between: compound 1b and iron(II), compound 2b and iron(II), compound 3b and iron(II), compound 4b and iron(II), compound 5b and iron(II), compound 6b and iron(II), compound 7b and iron(II), QF and iron(II), IQF and iron(II), QZF and iron(II), or QXF and iron(II).

In some embodiments, the present invention is directed to a method of inhibiting the proliferation of cancer cells in a subject in need thereof comprising administering an inhibitory amount of a compound of Formulas I-IV of the present invention to the subject. In some embodiments, the present invention is directed to a method of inhibiting the proliferation of cells arising from skin cancer, brain cancer, prostate cancer, ovarian cancer, breast cancer, thyroid cancer, liver cancer, stomach cancer, colon cancer, kidney cancer, pancreatic cancer, myeloma, multiple myeloma, leukemia, or a combination thereof.

In some embodiments, the present invention is directed to a method of treating a subject suffering from a hyperproliferative disorder comprising administering a therapeutically effective amount of a compound of Formulas I-IV of the present invention to the subject. In some embodiments, the present invention is directed to a method of treating a hyperproliferative disorder selected from: from skin cancer, brain cancer, prostate cancer, ovarian cancer, breast cancer, thyroid cancer, liver cancer, stomach cancer, colon cancer, kidney cancer, pancreatic cancer, myeloma, multiple myeloma, leukemia, and combinations thereof.

In some embodiments, the present invention is directed to a method of administering a therapeutically effective amount of a compound of Formulas I-IV to a subject suffering from a condition associated with an excess of a metal in the body. In some embodiments, the present invention is directed to a method of administering a compound of Formulas I-IV to a subject suffering from iron overload disorder and/or iron dysregulation. In some embodiments, the present invention is directed to a method of treating a disorder selected from: thalassemia, hemochromatosis, myelodysplasia, Alzheimer's disease, Parkinson's disease, and combinations thereof comprising administering a therapeutically effective amount of a compound of Formulas I-IV to the subject.

The compounds of the present invention are administered to a subject in need thereof in any manner that results in contact of the active agent with the agent's site of action. Administration that results in systemic bioavailability of the compounds of the present invention can be used, as well as local administration directly to a site on or in the body. The compounds of the present invention can be administered by conventional means available for use in conjunction with pharmaceuticals in a dosage range of 0.001 to 1000 mg/kg of mammal (e.g., human) bodyweight per day in a single dose or in divided doses. One dosage range is 0.01 to 500 mg/kg body weight per day orally in a single dose or in divided doses. Administration can be delivered as individual therapeutic agents or in a combination of therapeutic agents. They can be administered alone, but typically are administered with a pharmaceutically acceptable excipient selected on the basis of the chosen route of administration and standard pharmaceutical practice.

Compounds can be administered to a subject in one or more ways, including oral, parenteral (including subcutaneous injections, intravenous, intramuscular injection or infusion techniques), inhalation, buccal, sub lingual, or rectal, in the form of a unit dosage form that includes an effective amount of a compound of Formulas I-IV optionally in combination with one or more pharmaceutically-acceptable excipients. In some embodiments, the present invention is directed to a pharmaceutical composition comprising a compound of Formulas I-IV as disclosed herein. The pharmaceutical compositions of the present invention include one or compounds of Formulas I-IV and one or more pharmaceutically acceptable excipients. Pharmaceutical compositions containing the compound of the invention and a suitable carrier can be in various forms including, but not limited to, solids, solutions, powders, fluid emulsions, fluid suspensions, semi-solids, and dry powders including an effective amount of a compound of Formulas I-IV.

Pharmaceutically acceptable excipients include any excipient that can be compounded with a compound of Formulas I-IV, and includes but is not limited to bulking agents, fillers, carriers, adjuvants, vehicles, diluents, binders, disintegrants, stabilizers, anti-oxidants, lubricants, coatings, and other readily known excipients in standard pharmaceutical practice. Liquid preparations suitable for oral administration (e.g., suspensions, syrups, elixirs and other similar liquids) can contain media such as water, glycols, oils, alcohols, and the like. Solid preparations suitable for oral administration (e.g. powders, pills, capsules, and tablets) can contain solid excipients such as starches, sugars, kaolin, lubricants, binders, disintegrating agents, antioxidants and the like.

Parenteral compositions typically employ sterile water as a carrier and optionally other ingredients, such as solubility aids, stabilizers, anti-oxidants, tonicity agents, and the like. Injectable solutions can be prepared, for example, using a carrier comprising a saline solution, a glucose solution or a solution containing a mixture of saline and glucose. Further guidance for methods suitable for use in preparing pharmaceutical compositions is provided in Remington: The Science and Practice of Pharmacy, 21st edition (Lippincott Williams & Wilkins, 2006).

Therapeutic compounds can be administered orally in a dosage range of about 0.001 to 1000 mg/kg of mammal (e.g. human) body weight per day in a single dose or in divided doses. One dosage range is about 0.01 to 500 mg/kg body weight per day orally in a single dose or in divided doses. For oral administration, the compositions can be provided in the form of tablets or capsules containing about 1.0 mg to about 3,000 mg of the active ingredient. The specific dose level and frequency of dosage for any particular patient may be varied and will depend upon a variety of factors including the activity of the specific compound employed, the duration of action of that compound, the age, body weight, general health, sex, diet, mode and time of administration, rate of excretion, drug combination, the severity of the particular condition, and the host undergoing therapy. In view of the factors affecting the specific dose level and frequency it is contemplated that the dose frequency can range from multiple doses daily to monthly dosages. The preferred dose frequency ranges from once, twice or thrice per day to once every two weeks. A more preferred dose frequency ranges from once, twice or thrice per day to once weekly. A most preferred dose frequency ranges from once, twice or thrice per day to twice a week.

### Examples

The disclosure will now be illustrated with working examples, which are intended to illustrate the working of disclosure and not intended to restrict or limit the scope of the present disclosure. Unless defined otherwise, all technical and scientific terms used herein have the same meaning as commonly understood to one of ordinary skill in the art to which this disclosure belongs. Although other methods and materials similar or equivalent to those described herein can be used in the practice of the disclosed methods and compositions, the exemplary methods, devices and materials are described herein.

The reduction of tetrazolium cations was explored as a potential strategy to develop a new family of prochelators. Although the parent compound MTT presents low stability in blood serum¹⁶ and would not be ideal for in-vivo applications, synthetic modifications of the tetrazolium structure could increase its stability in biological environments and also enhance the metal-binding affinity of the formazan species resulting from the reduction/activation reaction.

When compared to other reductively activated prochelators (such as the disulfide-masked systems), this strategy based on tetrazolium cations offers at least two key advantages: 1) the positive charge and amphiphilicity of tetrazolium ions make them soluble in aqueous solutions and also capable to rapidly permeate cell membranes; and 2) the reduction potential of the tetrazolium ring can be carefully tuned via synthetic modification of its substituents, therefore the activation of the prochelators could be directed to specific reducing environments such as the cytoplasm of malignant cells.

### Preparation of Tetrazolium Compounds

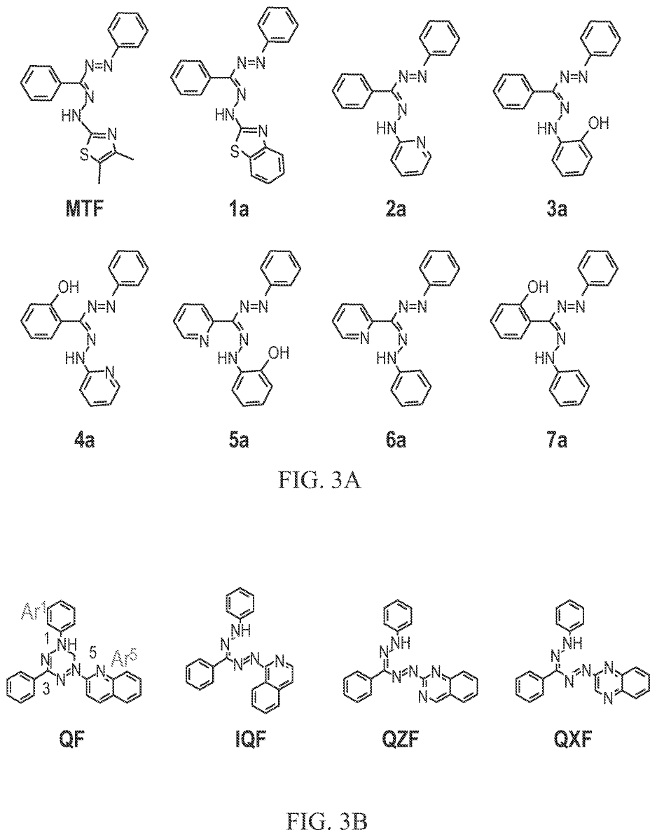

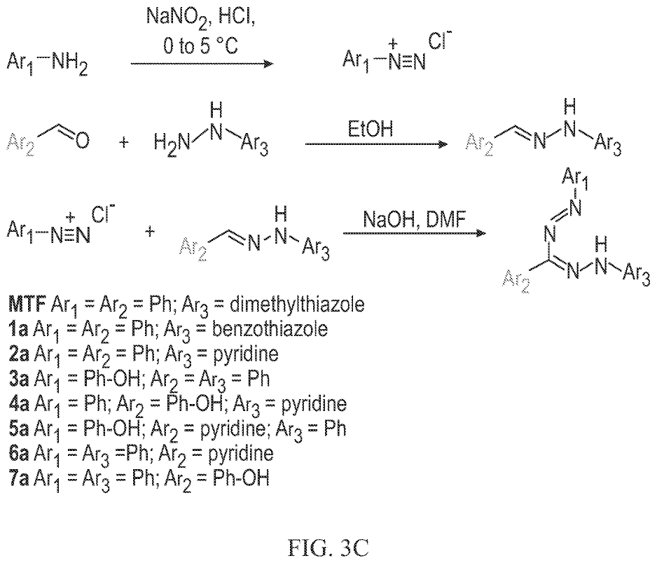

Representative formazan compounds of Formulas III and IV (FIGS. 3A-3B) were prepared by the process outlined in FIG. 3C. In some instances the compounds of FIGS. 3A-3B replace the thiazole heterocycle of MTT three metal-binding donors: benzothiazole, pyridine, or 2-hydroxyphenyl. In addition, changes on the central aryl group were introduced to assess their effect on the coordination mode of these formazans and/or on the reduction potential of the corresponding tetrazolium.

MTF and formazans 1a-5a were found to coordinate Fe(II) in buffered aqueous solutions as documented by changes in the optical absorption spectrum of the compounds particularly in the 500-800 nm region. In contrast, compounds 6a and 7a, which lack a metal-binding N-aryl group on the formazan scaffold did not show any indication of iron binding in the same conditions. These two compounds were retained in the experiments as controls.

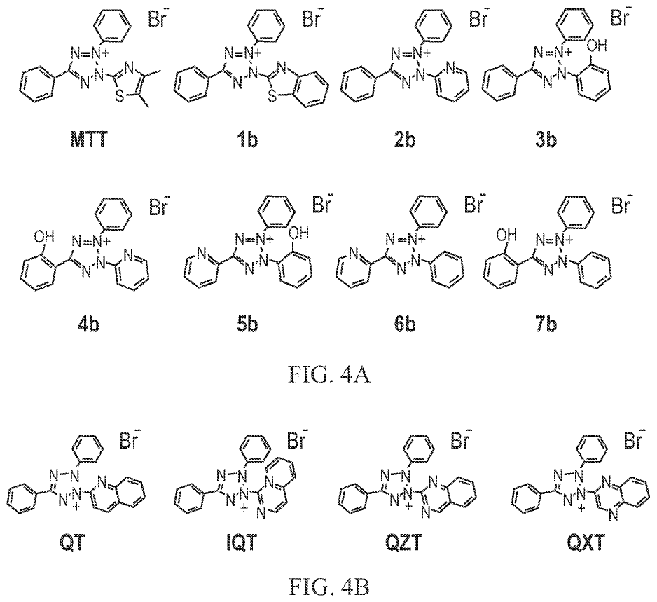

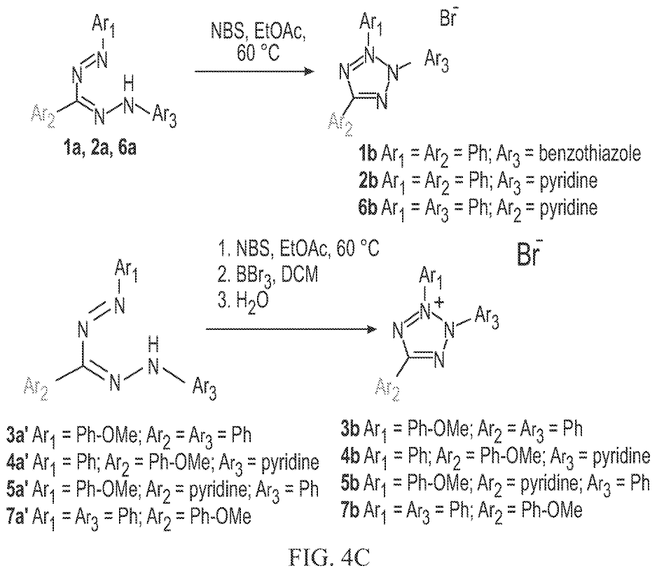

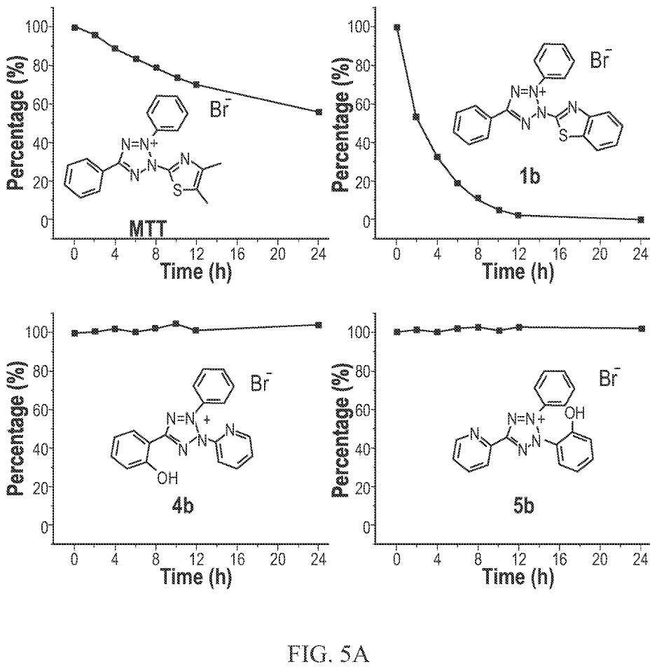

The tetrazolium salts (FIGS. 4A-4B) were synthesized from the formazans by the process outlined in FIG. 4C, which included oxidation with N-bromo-succinimide. For structures featuring a 2-hydroxyphenyl group, the oxidation was carried out on methoxyphenyl precursors and followed by a demethylation step in the presence of BBr₃. The tetrazolium compounds were isolated as bromide salts. As expected for compounds featuring a positively charged tetrazolium core, no indication of iron binding was observed in buffered aqueous solutions in the presence of Fe(II).

### Characterization of the Tetrazolium Compounds

The reduction of all the tetrazolium salts was investigated by cyclic voltammetry in buffered aqueous solutions (50 mM phosphate buffered saline solution, pH 7.4, K₃[Fe(CN)₆] as reference E₀=0.430 V vs. NHE, 25° C.). Each compound presents an irreversible reduction at a potential that varies considerably depending on the identity of the aryl substituents on the tetrazolium core. Although the peaks are rather broad and likely susceptible to shifts depending on the specific experimental conditions, the peak potentials (Table 1, referenced to NHE) of some of these tetrazolium compounds indicate that they are reduced outside the biologically relevant window. For instance, compounds 3b (Eₚ−328 mV) and 5b (Eₚ−380 mV) are not likely reduced in biological settings. On the other hand, the fact that the tetrazolium compounds are harder to reduce relative to MTT (with the exception of the benzothiazole derivative 1b) makes them well suited for intracellular activation in the reducing microenvironment of malignant cells. Compounds 2b, 4b, 6b and 7b are likely to be reduced intracellularly to the formazan species.

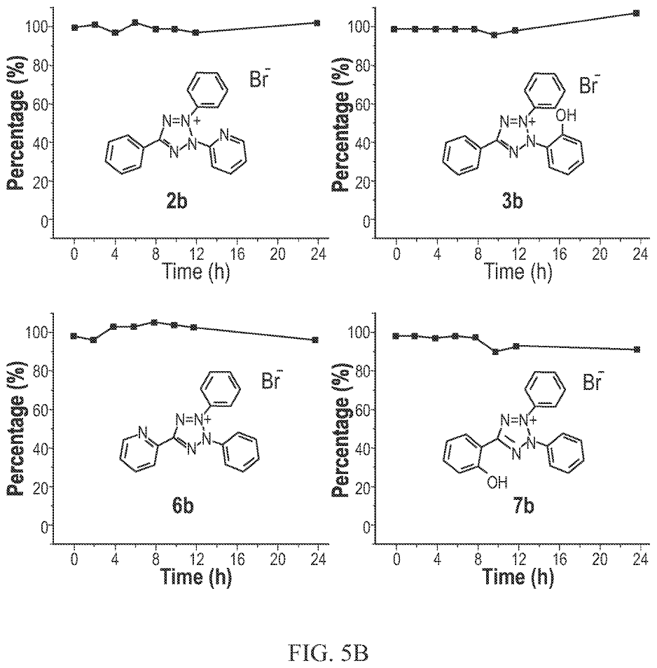

The stability of exemplary tetrazolium compounds was tested in fetal bovine serum and monitored by HPLC analysis. The results are displayed graphically in FIGS. 5A and 5B. Whereas the easily reduced MTT and benzothiazole-substituted 1b present significant degradation within the first 24 h, all the other tetrazolium ions (i.e., featuring phenyl, hydroxyphenyl and pyridyl groups) are completely stable in blood serum for 24 h.

### Antiproliferative Activity of the Tetrazolium Compounds

Once the stability data and the information on the reduction and iron binding of the new cohort of tetrazolium salts had been obtained, the antiproliferative activity of the new cohort was assessed in a panel of mammalian cell lines. Cancer cells require more iron than normal cells in order to sustain fast proliferation rate. Therefore, compounds that interfere with intracellular iron availability display antiproliferative activity.¹² Indeed iron chelation and prochelation strategies could provide new therapeutic avenues in anticancer drug discovery.⁴

Cancer cell lines of different origin were selected (ovarian A2780, breast MDAMB-231, colon Caco-2, and lung A549), and a normal cell line (MRC-5, lung fibroblasts) was selected as a comparison. Notably the importance of iron in cancer progression has been noted in multiple clinical studies for these malignancies.¹⁷⁻¹⁹

Because the colorimetric MTT reagent is one of the compounds being tested in this panel, the reduction of resazurin to brightly fluorescent resorufin was chosen as a readout for the assessment of antiproliferative activity. The IC50 values after 72-hour incubations are shown in Table 1.

TABLE 1
Reduction potentials and antiproliferative activities
of exemplary tetrazolium salts and formazan chelators.

| | | IC₅₀ Value (μM)* | | | | |
|---|---|---|---|---|---|---|
| | E_{red} vs. | A2780 | MDA-MB-231 | Caco-2 | A549 | MRC-5 |
| | NHE (mV) | (Ovary) | (Breast) | (Colon) | (Lung) | (Normal lung) |
| DFX | n.d. | 15 ± 0.5 | 17 ± 1 | 67 ± 2 | 21 ± 3 | >100 |
| MTT | 94 | 45 ± 9 | 48 ± 7 | 92 ± 7 | 44 ± 9 | 32 ± 5 |
| 1b | 207 | 23 ± 3 | 21 ± 2 | 40 ± 3 | 41 ± 3 | 97 ± 10 |
| 2b | −130 | 16 ± 3 | 16 ± 2 | 13 ± 2 | 17 ± 2 | >100 |
| 3b | −328 | >100 | >100 | >100 | >100 | >100 |
| 4b | −102 | 12 ± 3 | 17 ± 2 | 29 ± 3 | 28 ± 4 | >100 |
| 5b | −380 | 90 ± 10 | >100 | >100 | >100 | >100 |
| 6b | −241 | 74 ± 4 | >100 | >100 | >100 | >100 |
| 7b | −187 | 74 ± 7 | >100 | >100 | >100 | >100 |
| QT | n.d. | 0.8 ± 0.1 | 0.3 ± 0.1 | n.d. | n.d. | 3.5 ± 0.8 |
| QF | n.d. | 14 ± 2 | 7 ± 2 | n.d. | n.d. | 89 ± 9 |
| IQT | n.d. | 0.5 ± 0.1 | n.d. | n.d. | n.d. | 14 ± 2 |
| IQF | n.d. | 0.7 ± 0.1 | 1.4 ± 0.5 | n.d. | n.d. | n.d. |
| QZT | n.d. | n.d. | n.d | n.d. | n.d. | n.d. |
| QZF | n.d. | 0.6 ± 0.1 | 1.8 ± 0.5 | n.d. | n.d. | 4.2 ± 0.3 |
| QXT | n.d. | 19 ± 2 | 26 ± 3 | n.d. | n.d. | 45 ± 8 |
| QXF | n.d. | 9 ± 2 | 22 ± 3 | n.d. | n.d. | 11 ± 2 |

a Cell cultures were incubated with the tested compounds (0.032-100 μM) for 72 h; values are presented as mean ± SDM, n = 3; n.d.: not determined.

The iron chelator deferasirox (DFX) was used as a control compound known to display antiproliferative activity due to iron deprivation. This FDA-approved drug is clinically employed for the treatment of iron overload and has been tested in clinical trials as an anticancer agent.²⁰

In spite of their limited stability in serum, easily reduced MTT and 1b present moderate antiproliferative activities in the tested cancer cells. Conversely, compounds 6b and 7b, which are reduced to formazans that do not bind iron in aqueous solutions, show low toxicity throughout this cell panel. Among the pyridyl- and hydroxophenyl-substituted tetrazolium salts, compounds 2b and 4b, which are reduced at the most accessible potentials, present promising antiproliferative activities in the tested cancer cell lines. Furthermore, the proliferation of normal fibroblasts is not affected by these compounds (up to a 100 μM concentration) possibly owing to their lower susceptibility to iron deprivation and/or less reducing intracellular environment. Finally, compounds 3b and 5b, which are unlikely to be reduced in cells, present low cytotoxicity in this panel.

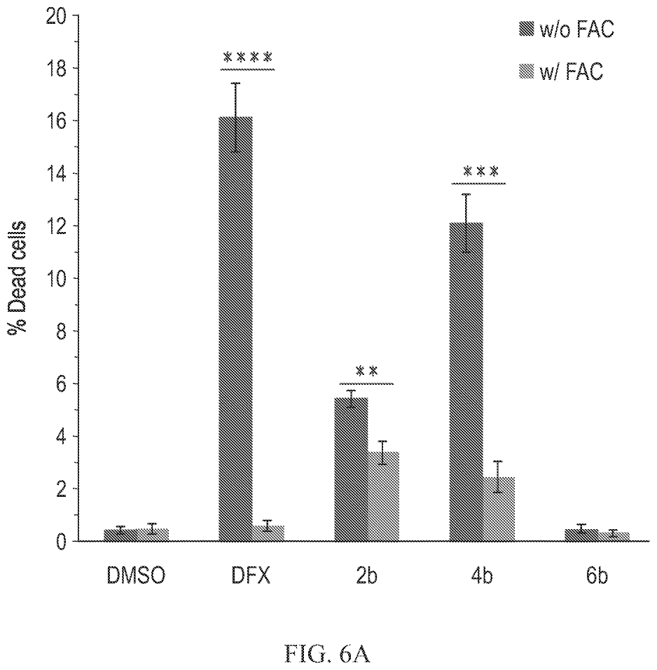

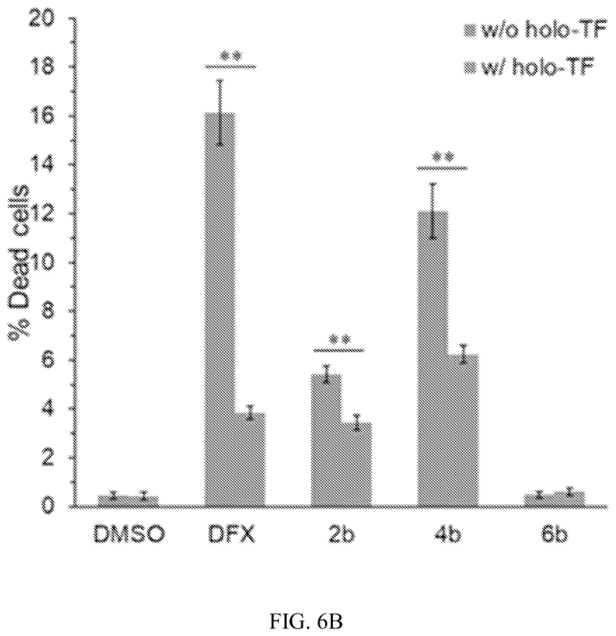

The biological activity of the tetrazolium and formazan compounds was investigated in A2780 cells, in which they presented the highest antiproliferative activity. In particular, experiments were performed to confirm that the antiproliferative activity of these compounds is associated with the availability of intracellular iron. Consistent with this expectation, A2780 cells exposed to 2b and 4b, as well as control chelator DFX, could be rescued by iron supplementation through incubation with ferric ammonium citrate or holotrasferrin (FIGS. 6A-6B). Conversely, the effects of 6b, which can be reduced in cells but does not bind iron, are not altered by iron supplementation.

A prochelation strategy was designed based on the reduction of tetrazolium ions, such as MTT, to metal-binding formazans in mammalian cells. Through chemical modification, a set of compounds was prepared with a variety of reduction potentials and metal-binding groups.

As expected for inactive prochelators, all the prepared tetrazolium cations do not bind iron in neutral aqueous solutions. Conversely, the formazan compounds with metal-binding pyridyl and hydroxyphenyl donors coordinate iron in aqueous solution.

Based on their chemical characterization and antiproliferative activities in a panel of human cell lines, the tetrazolium cations exhibited in vivo activity as prochelators based on their high stability in blood serum, capability of undergoing reduction at potentials that are accessible in the cellular milieu, and form iron-binding formazans upon reduction. Consistent with antiproliferative activities due at least in part to iron deprivation, cells exposed to the prochelators of the present invention are rescued in the presence of an iron source.

The tetrazolium compounds QT and IQT present sub-micromolar IC₅₀ values in A2780 cell lines. Notably, QT is also antiproliferative in triple negative breast cancer cells (MDA-MB-231), and both QT and IQT are less toxic to normal cells thereby showing promising therapeutic indexes. The formazan chelators QF, IQF, QZF and QXF are also generally antiproliferative at sub-micromolar or low-micromolar levels as expected for metal-binding species.

Overall, a new prochelation strategy was demonstrated to rely on the reductive activation of tetrazolium cations to form metal-binding formazan species upon cellular uptake. Because metal coordination is associated with optical absorption changes in the near-infrared region (600-800 nm), these compounds could also lead to the detection of metal ions via photoacoustic methods in biological settings. This new class of tetrazolium and formazan compounds could be employed in drug discovery and bioimaging efforts targeting the multifaceted role of transition metals in human health.

### REFERENCES

1. Richardson, D. R.; Kalinowski, D. S.; Lau, S.; Jansson, P. J.; Lovejoy, D. B., Cancer cell metabolism and the development of potent iron chelators as anti-tumor agents. Biochim. Biophys. Acta 2009, 1790, 120-111.
2. Torti, S. V.; Torti, F. M., Iron and cancer: more ore to be mined. Nat. Rev. Cancer 2013, 13, 342-355.
3. Zecca, L.; Youdim, M. B. H.; Riederer, P.; Connor, J. R.; Crichton, R. R., Iron, brain ageing and neurodegenerative disorders. Nat. Rev. Neurosci. 2004, 5, 863-873.
4. Kalinowski, D. S.; Richardson, D. R., The evolution of iron chelators for the treatment of iron overload disease and cancer. Pharmacol. Rev. 2005, 57, 547-583.
5. Liu, Z. D.; Hider, R. C., Design of iron chelators with therapeutic application. Coord. Chem. Rev. 2002, 232, 151-171.
6. Ba, L. A.; Doering, M.; Burkholz, T.; Jacob, C., Metal trafficking: from maintaining the metal homeostasis to future drug design. Metallomics 2009, 1, 292-311.
7. Perez, L. R.; Franz, K. J., Minding metals: Tailoring multifunctional chelating agents for neurodegenerative disease. Dalton Trans. 2010, 39, 2177-2187.
8. Wang, Q.; Franz, K. J., Stimulus-Responsive Prochelators for Manipulating Cellular Metals. Acc. Chem. Res. 2016, 49, 2468-2477.
9. Chang, T. M.; Tomat, E., Disulfide/thiol switches in thiosemicarbazone ligands for redoxdirected iron chelation. Dalton Trans. 2013, 42, 7846-7849.
10. Akam, E. A.; Chang, T. M.; Astashkin, A. V.; Tomat, E., Intracellular reduction/activation of a disulfide switch in thiosemicarbazone iron chelators. Metallomics 2014, 6, 1905-1912.
11. Akam, E. A.; Tomat, E., Targeting Iron in Colon Cancer via Glycoconjugation of Thiosemicarbazone Prochelators. Bioconjugate Chem. 2016, 27, 1807-1812.
12. Mertens, C.; Akam, E. A.; Rehwald, C.; Brune, B.; Tomat, E.; Jung, M., Intracellular Iron Chelation Modulates the Macrophage Iron Phenotype with Consequences on Tumor Progression. PROS ONE 2016, 11, eO 166164.
13. Akam, E. A.; Utterback, R. D.; Marcero, J. R.; Dailey, H. A.; Tomat, E., Disulfide-masked iron prochelators: Effects on cell death, proliferation, and hwmoglobin production. J. Inorg. Biochem. 2018, 180, 186-193.
14. Sung, Y.-S.; Wu, W.; Ewbank, M. A.; Utterback, R. D.; Marty, M. T.; Tomat, E., Albumin conjugates of thiosemicarbazone and imidazole-2-thione prochelators: Iron coordination and antiproliferative activity. ChemMedChem 2021, 16, 2764-2768.
15. Gilroy, J. B.; Often, E., Formazanate coordination compounds: synthesis, reactivity, and applications. Chem. Soc. Rev. 2020, 49, 85-113.
16. Funk, D.; Schrenk, H.-FL; Frei, E., Serum albumin leads to false-positive results in the XTT and the MTT assay. BioTechniques 2007, 43, 178-182.
17. Pinnix, Z. K.; Miller, L. D.; Wang, W.; D'Agostino, R.; Kute, T.; Willingham, M. C.; Hatcher, H.; Tesfay, L.; Sui, G.; Di, X.; Torti, S. V.; Torti, F. M., Ferroportin and Iron Regulation in Breast Cancer Progression and Prognosis. Sci. Transl. Med. 2010, 2, 43ra56.
18. Nelson, R. L., Iron and colorectal cancer risk: Human studies. Nutr. Rev. 2001, 59, 140-148.
19. Jung, M.; Mertens, C.; Tomat, E.; Briine, B., Iron as a Central Player and Promising Target in Cancer Progression. Int J Mol Sci 2019, 20, 273.
20. Bedford, M. R.; Ford, S. J.; Homiblow, R. D.; Iqbal, T. H.; Tselepis, C., Iron Chelation in the Treatment of Cancer: A New Role for Deferasirox?J. Clin. Pharmacol. 2013, 53, 885-891.

What is claimed is:

1. A method of inhibiting the proliferation of cancer cells in a subject in need thereof comprising administering an inhibitory amount of the compound of Formula I to the subject: 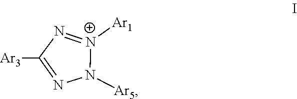
as a salt, hydrate, solvate, and/or complex,
wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

2. The method of claim 1, wherein Ar₁ is optionally substituted phenyl.

3. The method of any of claim 1 or 2, wherein Ar₃ is selected from: optionally substituted phenyl, optionally substituted 2-pyridyl, optionally substituted 2-thiazolyl, and optionally substituted 2-benzothiazolyl.

4. The method of any of claims 1-3, wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

5. The method of any of claims 1-4, wherein
Ar₁ is unsubstituted phenyl;
Ar₃ is selected from unsubstituted phenyl, 2-hydroxyphenyl, and 2-pyridyl; and
Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

6. The method of any of claims 1-4, wherein the compound of Formula I is selected from: 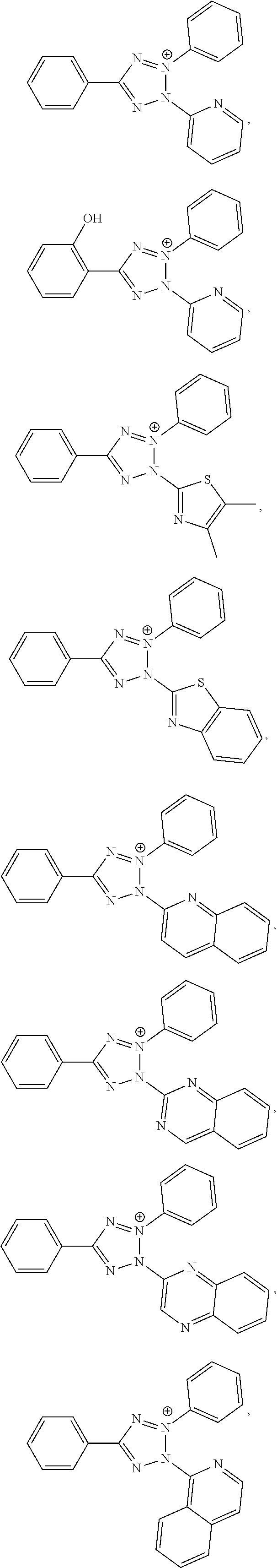
and combinations thereof.

7. The method of any of claims 1-6, wherein the compound of Formula I is administered as a bromide salt.

8. A method of treating a subject suffering from a hyperproliferative disorder comprising administering a therapeutically effective amount of the compound of Formula I to the subject: 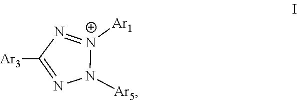
as a salt, hydrate, solvate, and/or complex,
wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

9. The method of claim 8, wherein Ar₁ is optionally substituted phenyl.

10. The method of any of claim 8 or 9, wherein Ar₃ is selected from: optionally substituted phenyl, optionally substituted 2-pyridyl, optionally substituted 2-thiazolyl, and optionally substituted 2-benzothiazolyl.

11. The method of any of claims 8-10, wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

12. The method of any of claims 8-11, wherein
Ar₁ is unsubstituted phenyl;
Ar₃ is selected from unsubstituted phenyl, 2-hydroxyphenyl, and 2-pyridyl; and
Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

13. The method of any of claims 8-11, wherein the compound of Formula I is selected from 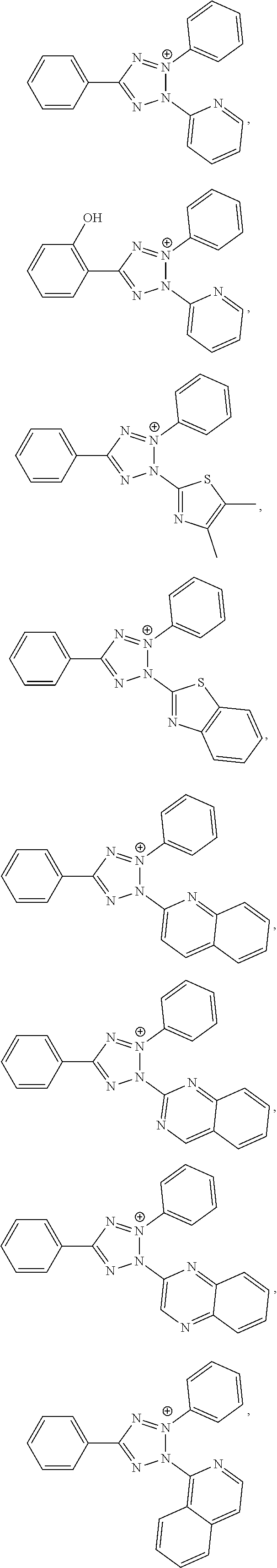
and combinations thereof.

14. The method of any of claims 8-13, wherein the compound of Formula I is administered as a bromide salt.

15. A method of sequestering iron in a subject in need thereof comprising administering a compound of Formula I to the subject: 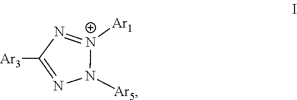
as a salt, hydrate, solvate, and/or complex,
wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof.

16. The method of claim 15, wherein Ar₁ is optionally substituted phenyl.

17. The method of any of claim 15 or 16, wherein Ar₃ is selected from: optionally substituted phenyl, optionally substituted 2-pyridyl, optionally substituted 2-thiazolyl, and optionally substituted 2-benzothiazolyl.

18. The method of any of claims 15-17, wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

19. The method of any of claims 15-18, wherein
Ar₁ is unsubstituted phenyl;
Ar₃ is selected from unsubstituted phenyl, 2-hydroxyphenyl, and 2-pyridyl; and
Ar₅ is selected from: unsubstituted phenyl, 2-hydroxylphenyl, unsubstituted 2-pyridyl, unsubstituted 2-quinolinyl, unsubstituted 1-isoquinolinyl, unsubstituted 2-quinazolinyl, unsubstituted 2-(1,4-benzo[a]pyrazine).

20. The method of any of claims 15-19, wherein the compound of Formula I is selected from: 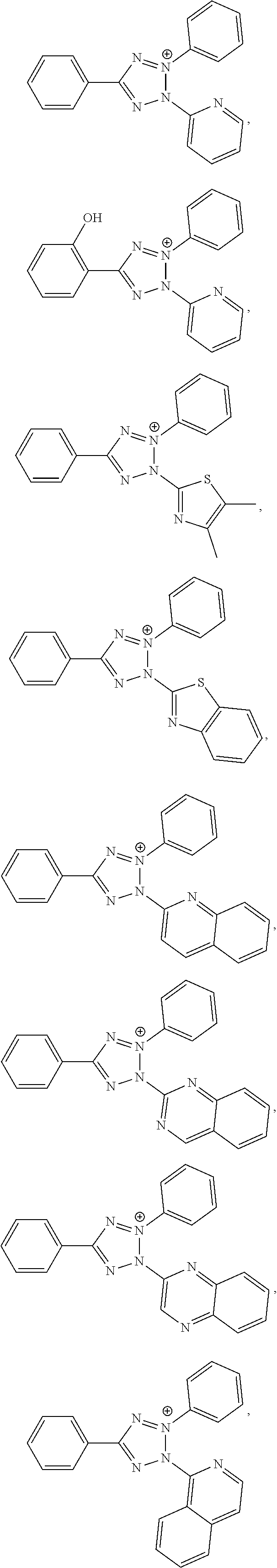
and combinations thereof.

21. The method of any of claims 15-20, wherein the compound of Formula I is administered as a bromide salt.

22. The method of any of claims 15-21, wherein the subject suffers from at least one disorder selected from: iron overload disorder, iron dysregulation, thalassemia, hemochromatosis, myelodysplasia, Alzheimer's disease, or Parkinson's disease.

23. A pharmaceutical composition comprising the a compound of Formula I 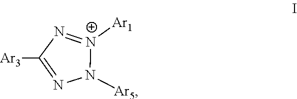
as a salt, hydrate, solvate, and/or complex,
wherein Ar₁, Ar₃ and Ar₅ are independently selected from: optionally substituted 2-pyrrolyl, optionally substituted 2-thiophenyl, optionally substituted 2-(1,3-thiazolyl), optionally substituted phenyl, optionally substituted 2-pyridyl, optionally substituted 3-pyridyl, optionally substituted 2-pyrimidinyl, optionally substituted 4-pyrimidinyl, optionally substituted 2-(1,3-benzothiazolyl), optionally substituted 1-naphthyl, optionally substituted 2-naphthyl, optionally substituted 2-quinolinyl, optionally substituted 3-quinolinyl, optionally substituted 1-isoquinolinyl, optionally substituted 3-isoquinolinyl, optionally substituted 2-quinazolinyl, optionally substituted 2-(1,4-benzo[a]pyrazine), optionally substituted with zero, one, two or three substituents selected from: methyl, trifluoromethyl, trichloromethyl, fluoro, chloro, bromo, cyano, carbonitrile, nitro, hydroxyl, amino, C₁-C₁₀ alkyl amino, ethyl, propyl, iso-propyl, C₁-C₁₀ ether, C₁-C₁₀ thioether, C₄-C₂₀ polyethyleneglycol, carboxyl, C₁-C₁₀ alkoxy, and combinations thereof, and
at least one pharmaceutically acceptable excipient.

24. The pharmaceutical composition of claim 23, wherein Ar₁ is unsubstituted phenyl.

25. The pharmaceutical composition of any of claim 23 or 24, wherein Ar₃ is selected from: optionally substituted phenyl, optionally substituted 2-pyridyl, optionally substituted 2-thiazolyl, and optionally substituted 2-benzothiazolyl.

26. The pharmaceutical composition of any of claims 23-25, wherein Ar₅ is selected from: optionally substituted phenyl, 2-hydroxyphenyl, optionally substituted 2-pyridyl, optionally substituted 2-quinolinyl, optionally substituted 3-quinolinyl, optionally substituted 1-isoquinolinyl, optionally substituted 2-quinazolinyl, and optionally substituted 2-(1,4-benzo[a]pyrazine).

27. The pharmaceutical composition of any of claims 23-26, wherein the compound of Formula I is selected from: 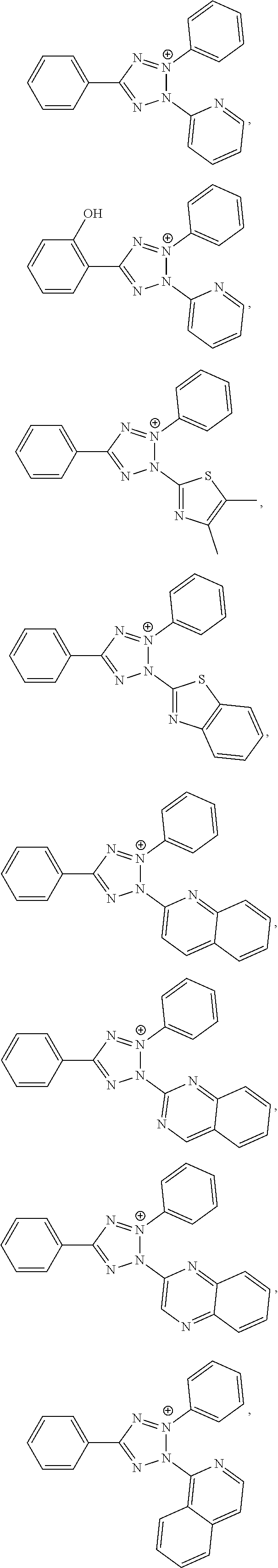
and combinations thereof.

28. The pharmaceutical composition of any of claims 23-27, wherein the compound of Formula I is present as a bromide salt.
